(19) United States
(12) Patent Application Publication
(10) Pub. No.: US 2022/0121854 A1
(43) Pub. Date: Apr. 21, 2022

(54) SYSTEMS AND METHODS FOR GENERATING VIDEO FILES OF DIGITAL X-RAY IMAGING

(71) Applicant: Illinois Tool Works Inc., Glenview, IL (US)

(72) Inventors: Jason William Bourn, Stratham, NH (US); Joseph Ryan Lapinskas, Windham, NH (US); Paul Benson, Waltham, MA (US); Daniel J. Jozwiak, Londonderry, NH (US)

(21) Appl. No.: 17/075,086

(22) Filed: Oct. 20, 2020

(51) Int. Cl. G06K 9/00; G06T 7/00; G06F 16/783

(52) U.S. Cl. CPC G06K 9/00744; G06T 7/0012; G06T 2207/10048; G06F 16/7837; G06T 2207/10116; G06K 9/00765

(57) ABSTRACT

An example method to generate a video from multiple cameras including a digital X-ray imaging device involves: initiating a recording session on an X-ray digital imaging device; capturing one or more images using a first one of a plurality of imaging sensors of the X-ray digital imaging device; capturing one or more videos using a second one of the plurality of imaging sensors; storing, on a machine readable storage device, two or more files corresponding to the one or more images and the one or more videos captured using the first and second ones of the plurality of imaging sensors; and combining the two or more files into a single video file representative of the recording session.

### BACKGROUND

This disclosure relates generally to radiography and, more particularly, to systems and methods for generating video files of digital X-ray imaging.

### SUMMARY

Systems and methods for digital X-ray imaging are disclosed, substantially as illustrated by and described in connection with at least one of the figures, as set forth more completely in the claims.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a perspective view of an example handheld X-ray imaging system to generate and output digital images and/or video based on incident X-rays, in accordance with aspects of this disclosure.

FIG. 2 is a block diagram of the example handheld X-ray imaging system of FIG. 1.

FIG. 3 is a perspective view of a first portion of the handheld X-ray imaging system of FIG. 1, including an X-ray generator, a power supply, an operator control handle.

FIG. 4 is a more detailed view of the first portion of the handheld X-ray imaging system of FIG. 3 including the example handle.

FIGS. 5A and 5B illustrate perspective views of the example handle of FIG. 3.

FIG. 6 is a partially exploded view of the example digital X-ray detector of FIG. 1.

FIG. 7 is a perspective view of a first portion of the handheld X-ray imaging system of FIG. 1, including a digital X-ray detector assembly.

FIG. 8 is a side view of the example digital detector housing, the scintillator, and the reflector.

FIG. 9 is a side view of the example digital X-ray detector of FIG. 1, illustrating imaging of incident X-rays by the digital X-ray detector.

FIG. 10 is a side view of the handheld X-ray imaging system of FIG. 1, illustrating scanning of an object under test by directing X-rays from the X-ray tube to the X-ray detector.

FIGS. 11A and 11B illustrate example images and videos captured and stored, by the example handheld X-ray imaging system of FIG. 1, as files in association with recording sessions.

FIG. 12 illustrates an example of combining multiple files containing images and/or videos into a single, seamless video file captured by the example handheld X-ray imaging system of FIG. 1.

FIGS. 13A and 13B illustrate a flowchart representative of example machine readable instructions which may be executed by the example computing device of FIG. 2 to capture and store image(s) and/or video(s).

FIG. 14 is a flowchart representative of example machine readable instructions which may be executed by the example computing device of FIG. 2 to combine files into a single video file representative of a recording session.

FIG. 15 is a block diagram of an example computing system that may be used to implement the computing device and/or the external computing device of FIG. 2.

The figures are not necessarily to scale. Wherever appropriate, similar or identical reference numerals are used to refer to similar or identical components.

### DETAILED DESCRIPTION

Disclosed example handheld X-ray imaging systems enable real-time generation and/or display of digital images during X-ray radiography. In contrast with conventional systems, disclosed examples provide an all-in-one X-ray radiography unit that does not require extraneous equipment for power, X-ray generation, radiograph capture, radiograph display, or radiograph storage.

Disclosed example handheld X-ray imaging systems reduce operator fatigue relative to conventional scanning devices by having a reduced weight (e.g., less than 20 pounds) and/or by providing improved weight distribution that concentrates the weight of the handheld X-ray imaging system near the operator's body.

Additionally, relative to conventional X-ray scanning systems, disclosed systems and methods provide an easier method to access recordings of X-ray scanning of objects. In many use cases, the operator may use a targeting camera to correctly position or verify the position of the X-ray beam with reference to the object, and then activate the X-ray output to perform X-ray scanning. While conventional, analog imaging systems output streams of analog video to a recording or display device, disclosed example systems and methods enable a seamless digital video representative of the scanning procedure to be generated by combining multiple image and/or video files captured using multiple imaging sensors into a single video file. This video file may be used for subsequent analysis of the scanned object and/or any other purpose.

As used herein, the term “real-time” refers to the actual time elapsed in the performance of a computation by a computing device, the result of the computation being required for the continuation of a physical process (i.e., no significant delays are introduced). For example, real-time display of captured images includes processing captured image data and displaying the resulting output images to create the perception to a user that the images are displayed immediately upon capture. As used herein, the term “portable” includes handheld (e.g., capable of being carried and operated by a single person) and/or wheeled (e.g., capable of being transported and operated while wheels are attached and/or placed on wheels).

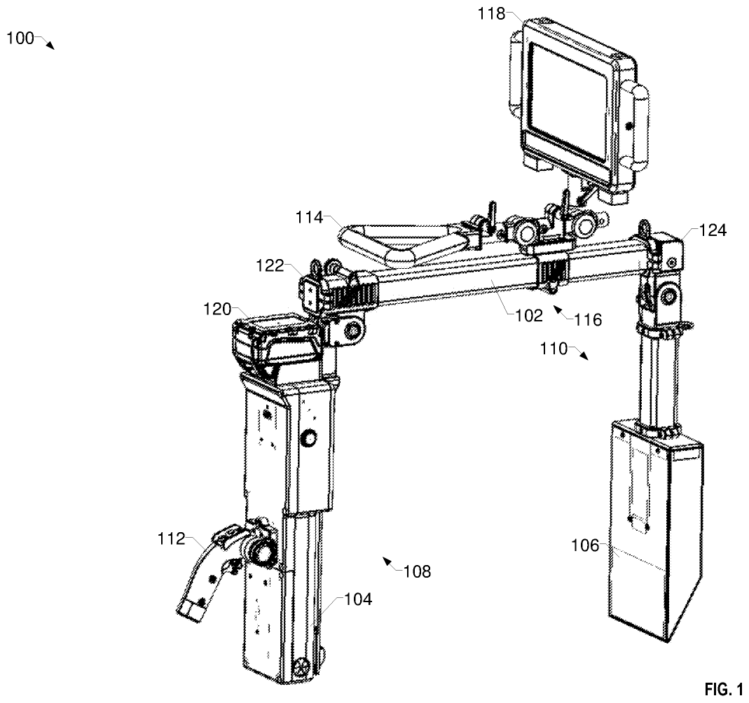

FIG. 1 is a perspective view of an example handheld X-ray imaging system 100 to generate and output digital images and/or video based on incident X-rays. The example handheld X-ray imaging system 100 may be used to perform non-destructive testing (NDT), medical scanning, security scanning, and/or any other scanning application.

The system 100 of FIG. 1 includes a frame 102 that holds an X-ray generator 104 and an X-ray detector 106. In the example of FIG. 1, the frame 102 is C-shaped, such that the X-ray generator 104 directs X-ray radiation toward the X-ray detector 106. As described in more detail below, the frame 102 is positionable (e.g., held by an operator, supported by an external support structure and/or manipulated by the operator, etc.) around an object to be scanned with X-rays. The example frame 102 is constructed using carbon fiber and/or machined aluminum.

The X-ray generator 104 is located on a first section 108 of the C-shaped frame 102 generates and outputs X-ray radiation, which traverses and/or scatters based on the state of the object under test. The X-ray detector 106 is located on a second section 110 of the frame 102 (e.g., opposite the first section 108) and receives incident radiation generated by the X-ray generator 104.

The example frame 102 may be manipulated using one or more handles 112, 114. A first one of the handles 112 is an operator control handle, and enables an operator to both mechanically manipulate the frame 102 and control the operation of the handheld X-ray imaging system 100. A second one of the handles 114 is adjustable and may be secured to provide the operator with leverage to manipulate the frame 102. The example handle 114 may be oriented with multiple degrees of freedom and/or adjusted along a length of a central section 116 of the frame 102.

During operation, the handheld X-ray imaging system 100 generates digital images (e.g., digital video and/or digital still images) from the X-ray radiation. The handheld X-ray imaging system 100 may store the digital images on one or more storage devices, display the digital images on a display device 118, and/or transmit the digital images to a remote receiver. The example display device 118 is attachable to the example frame 102 and/or may be oriented for viewing by the operator. The display device 118 may also be detached from the frame 102. When detached, the display device 118 receives the digital images (e.g., still images and/or video) via a wireless data connection. When attached, the display device 118 may receive the digital images via a wired connection and/or a wireless connection.

A power supply 120, such as a detachable battery, is attached to the frame 102 and provides power to the X-ray generator 104, the X-ray detector 106, and/or other circuitry of the handheld X-ray imaging system 100. An example power supply 120 that may be used is a lithium-ion battery pack. The display device 118 may receive power from the power supply 120 and/or from another power source such as an internal battery of the display device 118.

The example central section 116 of the frame 102 is coupled to the first section 108 via a joint 122 and to the second section 110 via a joint 124. The example joints 122, 124 are hollow to facilitate routing of cabling between the sections 108, 110, 116. The joints 122, 124 enable the first section 108 and the second section 110 to be folded toward the center section to further improve the compactness of the handheld X-ray imaging system 100 when not in use (e.g., during storage and/or travel).

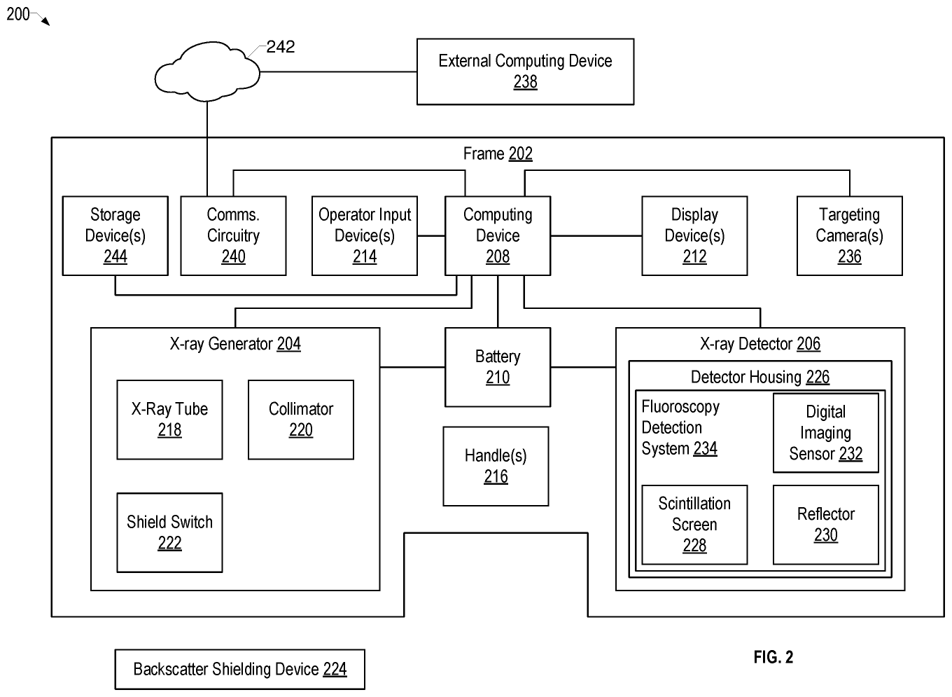

FIG. 2 is a block diagram of an example digital X-ray imaging system 200 that may be used to implement the handheld X-ray imaging system 100 of FIG. 1. The example digital X-ray imaging system 200 of FIG. 2 includes a frame 202 holding an X-ray generator 204, an X-ray detector 206, a computing device 208, a battery 210, one or more display device(s) 212, one or more operator input device(s) 214, and one or more handle(s) 216.

The X-ray generator 204 includes an X-ray tube 218, a collimator 220, and a shield switch 222. The X-ray tube 218 generates X-rays when energized. In some examples, the X-ray tube 218 operates at voltages between 40 kV and 120 kV. In combination with a shielding device, X-ray tube voltages between 70 kV and 120 kV may be used while staying within acceptable X-ray dosage limits for the operator. Other voltage ranges may also be used.

The collimator 220 filters the X-ray radiation output by the X-ray tube 218 to more narrowly direct the X-ray radiation at the X-ray detector 206 and any intervening objects. The collimator 220 reduces the X-ray dose to the operator of the system 200, reduces undesired X-ray energies to the detector 206 resulting from X-ray scattering, and/or improves the resulting digital image generated at the X-ray detector 206.

The shield switch 222 selectively enables and/or disables the X-ray tube 218 based on whether a backscatter shielding device 224 is attached to the frame. The backscatter shielding device 224 reduces the dose to the operator holding the frame 202 by providing shielding between the collimator 220 and an object under test. The example backscatter shielding device 224 includes a switch trigger configured to trigger the shield switch 222 when properly installed. For example, the shield switch 222 may be a reed switch or similar magnetically-triggered switch, and the backscatter shielding device 224 includes a magnet. The reed switch and magnet are respectively positioned on the frame 202 and the backscatter shielding device 224 such that the magnet triggers the reed switch when the backscatter shielding device 224 is attached to the frame 202. The shield switch 222 may include any type of a capacitive sensor, an inductive sensor, a magnetic sensor, an optical sensor, and/or any other type of proximity sensor.

The shield switch 222 is configured to disable the X-ray tube 218 when the backscatter shielding device 224 is not installed. The shield switch 222 may be implemented using, for example, hardware circuitry and/or via software executed by the computing device 208. In some examples, the computing device 208 may selectively override the shield switch 222 to permit operation of the X-ray tube 218 when the backscatter shielding device 224 is not installed. The override may be controlled by an administrator or other authorized user.

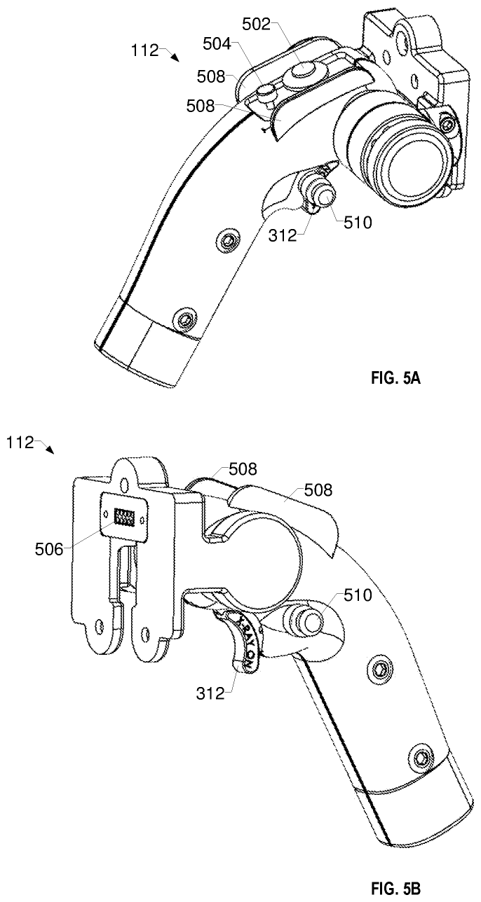

The X-ray detector 206 of FIG. 2 generates digital images based on incident X-ray radiation (e.g., generated by the X-ray tube 218 and directed toward the X-ray detector 206 by the collimator 220). The example X-ray detector 206 includes a detector housing 226, which holds a scintillation screen 228, a reflector 230, and a digital imaging sensor 232. The scintillation screen 228, the reflector 230, and the digital imaging sensor 232 are components of a fluoroscopy detection system 234. The example fluoroscopy detection system 234 is configured so that the digital imaging sensor 232 (e.g., a camera, a sensor chip, etc.) receives the image indirectly via the scintillation screen 228 and the reflector 230. In other examples, the fluoroscopy detection system 234 includes a sensor panel (e.g., a CCD panel, a CMOS panel, etc.) configured to receive the X-rays directly, and to generate the digital images. An example implementation of the X-ray detector 206 is described below with reference to FIGS. 5-8.

In some other examples, the scintillation screen 228, may be replaced with a solid state panel that is coupled to the scintillation screen 228 and has pixels that correspond to portions of the scintillation screen 228. Example solid state panels may include CMOS X-ray panels and/or CCD X-ray panels.

The computing device 208 controls the X-ray tube 218, receives digital images from the X-ray detector 206 (e.g., from the digital imaging sensor 232), and outputs the digital images to the display device 212. Additionally or alternatively, the computing device 208 may store digital images to a storage device. The computing device 208 may output the digital images as digital video to aid in real-time non-destructive testing and/or store digital still images.

As mentioned above, the computing device 208 may provide the digital images to the display device(s) 212 via a wired connection or a wireless connection. To this end, the computing device 208 includes wireless communication circuitry. For example, the display device(s) 212 may be detachable from the frame 202 and held separately from the frame 202 while the computing device 208 wirelessly transmits the digital images to the display device(s) 212. The display device(s) 212 may include a smartphone, a tablet computer, a laptop computer, a wireless monitoring device, and/or any other type of display device equipped with wired and/or wireless communications circuitry to communicate with (e.g., receive digital images from) the computing device 208.

In some examples, the computing device 208 adds data to the digital images to assist in subsequent analysis of the digital images. Example data includes a timestamp, a date stamp, geographic data, or a scanner inclination. The example computing device 208 adds the data to the images by adding metadata to the digital image file(s) and/or by superimposing a visual representation of the data onto a portion of the digital images.

The operator input device(s) 214 enable the operator to configure and/or control the example digital X-ray imaging system 200. For example, the operator input device(s) 214 may provide input to the computing device 208, which controls operation and/or configures the settings of the digital X-ray imaging system 200. Example operator input device(s) 214 include a trigger (e.g., for controlling activation of the X-ray tube 218), buttons, switches, analog joysticks, thumbpads, trackballs, and/or any other type of user input device.

The handle(s) 216 are attached to the frame 202 and enable physical control and manipulation of the frame 202, the X-ray generator 204, and the X-ray detector 206. In some examples, one or more of the operator input device(s) 214 are implemented on the handle(s) 216 to enable a user to both physically manipulate and control operation of the digital X-ray imaging system 200.

The example digital X-ray imaging system 200 further includes one or more targeting camera(s) 236. The targeting camera(s) 236 output image(s) and/or videos, such as to the display device(s) 212, that enable an operator to view the target that will be imaged using the X-ray detector 206 when the trigger or other input device activates output of the X-rays. For example, the targeting camera(s) 236 may have a field of view that overlaps with the exposure field of the X-ray tube 218, such that the targeting camera(s) 236 generate image(s) and/or video(s) representing the object in the field of exposure of the X-ray tube 218. The targeting camera(s) 236 may include visible light cameras, infrared cameras, thermal imaging cameras, and/or any other type of camera or imaging sensor. In the example digital X-ray imaging system 200, the targeting camera(s) 236 may be located on a same portion of the frame 202 as the X-ray generator 204.

The example digital X-ray imaging system 200 may communicate with one or more external computing device(s) 238, such as to transfer image(s) and/or video(s) captured and/or stored by the digital X-ray imaging system 200 for storage and/or processing. Example external computing device(s) 238 include personal computers, laptops, servers, tablet computers, smartphones, and/or any other type of computing device. To facilitate communication, the example digital X-ray imaging system 200 includes communication circuitry 240, which may communicate directly with the external computing device(s) 238 (e.g., via wired or wireless communication) and/or via one or more communication networks 242. In some example, the external computing device 238 includes a display (e.g., the display device(s)) to view the image(s) and/or video(s) in real-time as they are transmitted from the digital X-ray imaging system 200.

In some examples, the digital X-ray imaging system 200 enables the operator to combine a stream of image(s) and/or video(s) from both the targeting camera(s) 236 and the X-ray detector 206 into a single, contiguous video. Videos from the targeting camera(s) 236 and the X-ray detector 206 may be combined or stitched together by the computing device 208, as part of a synchronization process with the external computing device 238, during processing of uploaded image(s) and/or video(s) at the external computing device 238, and/or at any other time. The combined video(s) are then saved and can be viewed on the external computing device(s) 238, transmitted to other computing device for viewing by the appropriate personnel, and/or stored on a storage device 244 of the digital X-ray imaging system 200 and/or a storage device of the external computing device 238 for subsequent use and/or transmission.

In an example of operation, an operator of the digital X-ray imaging system 200 may start a recording session on the digital X-ray imaging system 200, at which time the computing device 208 generates a new directory (e.g., based on a time stamp) to store image files and/or video files captured during the recording session. For example, the operator may start and/or end a recording session using one or more of the operator input device(s) 214, a remote control device, and/or using any other form of input. Additionally or alternatively, a recording session may be automatically started and/or automatically ended in response to predetermined events and/or criteria.

A recording session will be stored as one, two, three, or more separate files depending on the events occurring during the recording session. For example, the recording session will include one file if the user starts and stops the recording session while remaining on either the targeting camera(s) 236 or the X-ray detector 206 for the duration of the recording session. The recording session will include two files if the operator starts the recording while using the X-ray detector 206 and then releases the trigger (or other input device controlling the X-ray output) and stops the recording session while using the targeting camera(s) 236, or if the user starts the recording while using the targeting camera(s) 236 and presses the trigger (or other input device controlling the X-ray output), and then stops the recording session while using the X-ray detector 206. The recording session will include three files if, for example, if the user starts the recording while using the targeting camera(s) 236 and presses the trigger (or other input device controlling the X-ray output) and later releases the trigger while continuing the recording session. The recording session may include more than three files if, for example, the user presses and releases the trigger to switch between the targeting camera(s) 236 and the X-ray detector 206 multiple times during the same recording session.

At a later time, such as in response to a synchronization process in which stored files are transmitted to the external computing device 238, each recording session involving multiple files is combined into a single, contiguous video of the recording session. As a result, the image(s) and/or video(s) from multiple cameras or imaging sensors can be easily viewed. In some examples, to combine multiple image and/or video files into a single video file, the external computing device 238 identifies recording sessions (e.g., folder file structures) having multiple image and/or video files. Upon identifying a multi-file recording session, the external computing device 238 splits up each video file into individual frames, and saves those frames to files with a sequential file name (e.g., frame1, frame2, etc.) into a working directory. After splitting up all of the video files, the working directory contains images and individual video frames from all of the videos of the recording session. The external computing device 238 resumes the frame file numbering for each successive video where the numbering left off for the previous video file. The external computing device 238 then combines the successive frames in sequence to create the video and store in a single video file. The external computing device 238 may further delete the original files used to create the combined video.

The resulting video is an easily viewable representation of an X-ray imaging session that may involve the targeting camera(s) 236 showing the visible and/or invisible aspects used to recognize targets for scanning, and/or the X-ray imaging showing the scanning of the interior of the target object.

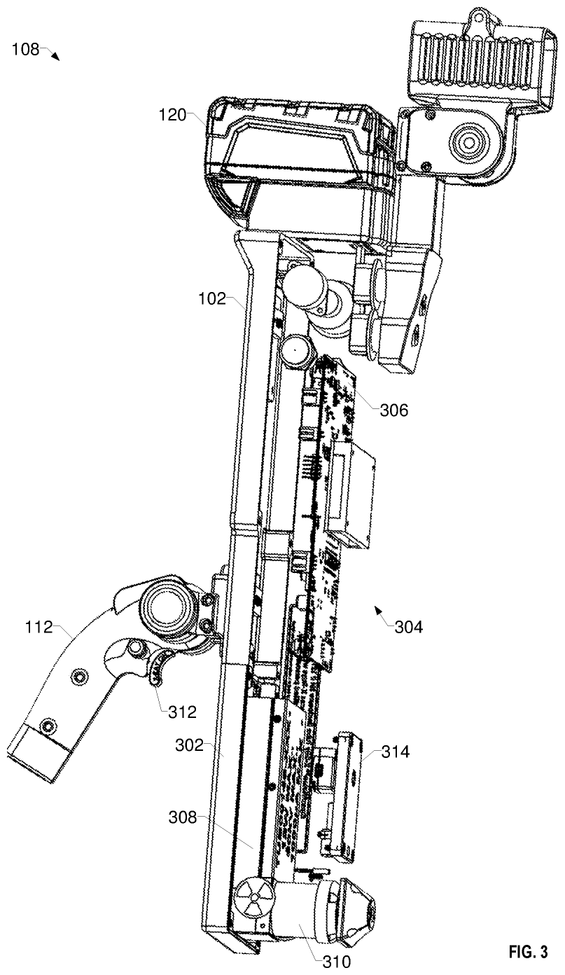

FIG. 3 is a perspective view of the first section 108 of the handheld X-ray imaging system 100 of FIG. 1, including the X-ray generator 104, the power supply 120, and the operator control handle 112. FIG. 3 is illustrated with a portion of a housing 302, while a second portion of the housing (shown in FIG. 1) is omitted for visibility of other components.

The example first section 108 is further coupled to a computing device 304, such as the computing device 208 of FIG. 2. The computing device 304 is attached to the frame 102 via a printed circuit board 306.

An X-ray tube 308 (e.g., the X-ray tube 218 of FIG. 2) is coupled to a collimator 310 (e.g., the collimator 220 of FIG. 2) and controlled by the computing device 304 and/or by an operator input device on the handle 112. As shown in FIG. 3, the handle 112 may include an X-ray trigger 312 (e.g., one of the operator input device(s) 214 of FIG. 2). When actuated (e.g., by the operator of the handheld X-ray imaging system 100), the X-ray trigger 312 activates the X-ray tube 308 to generate X-ray radiation. The X-ray trigger 312 may activate the X-ray tube 308 directly and/or via the computing device 304.

The collimator 310 filters X-ray radiation generated by the X-ray tube 308 to reduce the X-ray radiation that is not directed at the X-ray detector 106 and/or to increase the proportion of X-ray radiation that is directed at the X-ray detector 106 (e.g., radiation that ends up being incident on a scintillator of the X-ray detector 106) relative to radiation not directed at the X-ray detector 106.

A targeting camera 314 (e.g., the targeting camera 236 of FIG. 2) is coupled to the computing device 304 to enable an operator of the handheld X-ray imaging system 100 to determine a target of generated X-rays. The example targeting camera 314 generates and outputs digital images (e.g., digital video, digital still images, etc.) to the computing device 304 for display to the operator via the display device 118. The digital images of the target (e.g., an exterior of the target) may be saved in association with the digital images of the X-ray scanning to provide contextual information about the location or object from which digital X-ray images are captured. Additionally or alternatively, a laser may be projected from the location of the targeting camera 314 toward the X-ray detector 106. The laser illuminates an approximate location on a workpiece that is being scanned by the digital X-ray imaging system 100 and/or output to the display device 118.

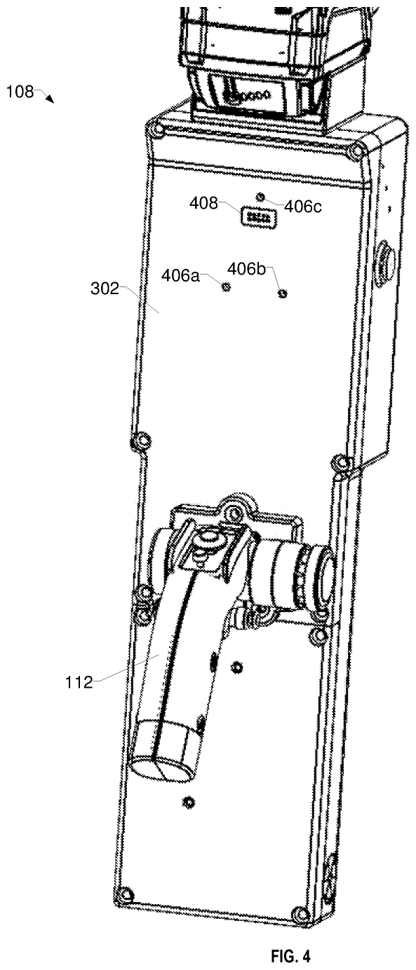

FIG. 4 is a more detailed view of the first section 108 of the handheld X-ray imaging system of FIG. 3 including the example handle 112. To improve the handling of the digital X-ray imaging system 100, the handle 112 is capable of attachment to multiple locations on the frame 102. The handle 112 is illustrated at a first location 402 on the frame 102 in FIG. 4. In the example of FIG. 4, the handle 112 is secured to the housing 302 via multiple screws.

The handle 112 may be detached from the first location 402 and attached at a second location 404. As illustrated in FIG. 4, the second location 404 on the housing 302 includes multiple screw nuts 406a-406c and a data connector 408, which match screw nuts and a data connector at the first location 402. The example handle 112 may be attached to the second location 404 by connecting a corresponding connector on the handle 112 to the data connector 408 and screwing the handle into the screw nuts 406a-406c.

FIGS. 5A and 5B illustrate perspective views of the example handle 112 of FIGS. 1 and 3. As mentioned above, the handle 112 includes the trigger 312, which enables and/or activates the X-ray tube 308 to output the X-ray radiation. The handle 112 includes additional input devices 502, 504 (e.g., operator input devices 214 of FIG. 2). The input device 502 is a thumbstick, which can be used to input commands to the computing device 304, such as navigating menus, confirming selections, configuring the X-ray tube 308 and/or the X-ray generator 104, changing views and/or any other type of operator input. The input device 504 is a push button that may be used by an operator to confirm and/or cancel a selection. The computing device 304 controls the X-ray tube 308, the X-ray detector 106 (e.g., the X-ray generator 204 and/or the digital imaging sensor 232 of FIG. 2), the display device 118, and/or any other aspect of the digital X-ray imaging system 100 based on input from the trigger 312, the input devices 502, 504, and/or any other input devices.

The handle 112 includes a data connector 506, which mates to the data connector(s) 408 on the housing 302. The data connectors 408, 506 establish a hard-wired connection between the trigger 312 and/or the input devices 502, 504 and the computing device 304 and/or other circuitry.

The handle 112 includes input guards 508, which protect the input devices 502, 504 from accidental damage. The input guards 508 extend from the handle 112 adjacent the input devices 502, 504 and farther than the input devices 502, 504.

The example handle 112 further includes a trigger lock 510. The trigger lock 510 is a mechanical lock that, when activated, mechanically prevents activation of the trigger 312. The example trigger lock 510 is a push-button safety that locks the trigger 312 against depression by the operator.

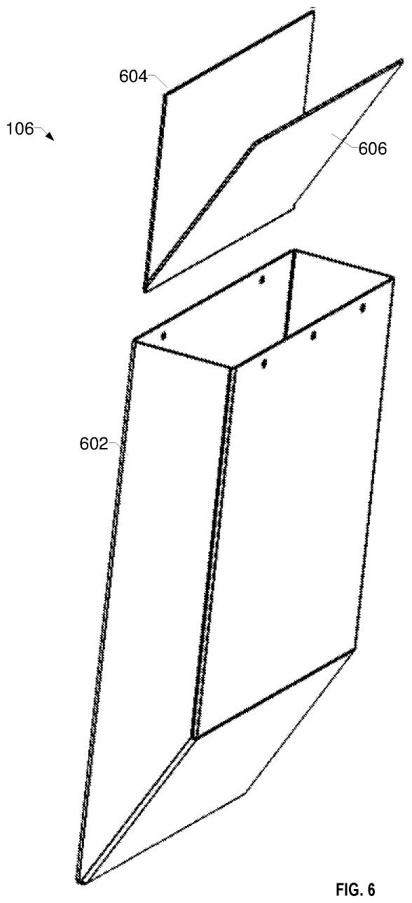

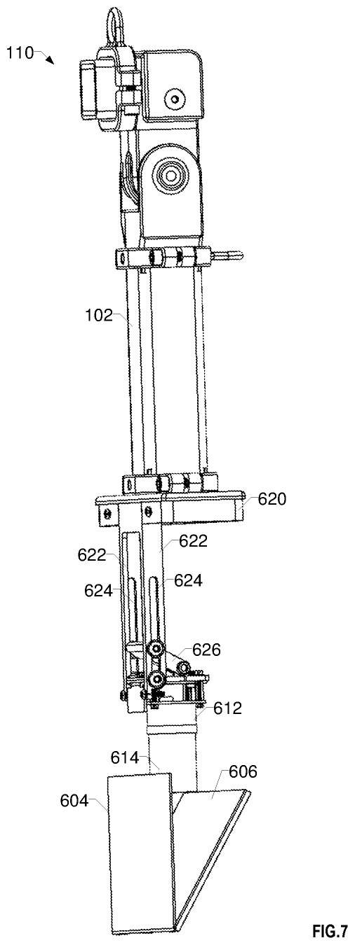

FIG. 6 is a partially exploded view of the example digital X-ray detector 106 of FIG. 1. FIG. 7 is a perspective view of the example digital X-ray detector 106 of FIG. 1. As illustrated in FIG. 6, the X-ray detector 106 includes a detector housing 602, a scintillation screen 604, and a reflector 606. The scintillation screen 604 and the reflector 606 are held within the housing 602 and are illustrated in FIG. 6 to show the relationship between the shape of the housing 602 and the geometries of the scintillation screen 604 and the reflector 606.

The detector housing 602 may be constructed using carbon fiber, aluminum, and/or any other material and/or combination of materials. The example detector housing 602 may function as a soft X-ray filter to reduce undesired X-ray radiation at the scintillation screen 604, thereby reducing noise in the resulting digital image. The scintillation screen 604 and/or the reflector 606 may be attached to the detector housing 602 using adhesive (e.g., epoxy, glue, etc.) and/or any other attachment technique. In some examples, the detector housing 602 is lined with a layer of lead or another backscatter shielding material to lower the dose to the operator in a handheld system.

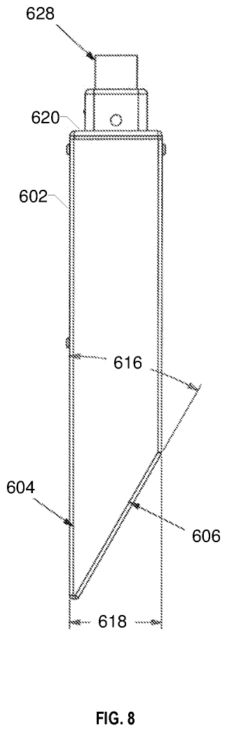

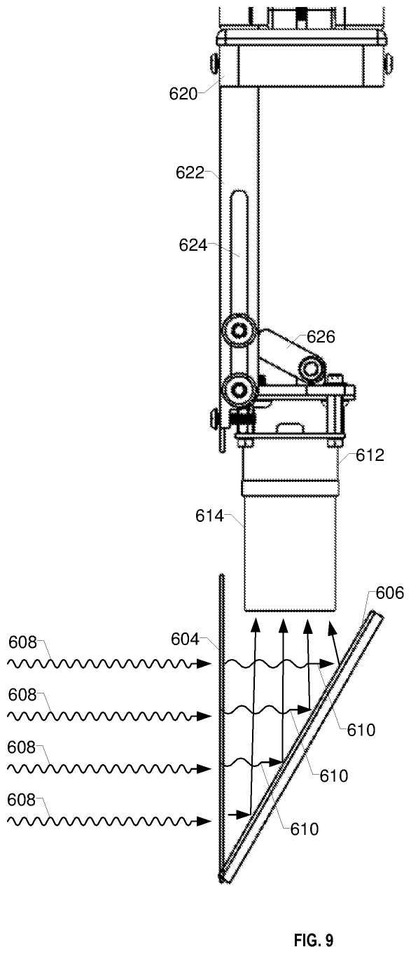

FIG. 8 is a side view of the example digital detector housing, the scintillator, and the reflector. FIG. 9 is a side view of the example digital X-ray detector 106 of FIG. 1, illustrating imaging of incident X-rays by the digital X-ray detector. As illustrated in FIG. 9, a digital imaging sensor 612 is oriented to capture light generated by the scintillation screen 604 in response to incident X-ray radiation.

The scintillation screen 604 converts incident X-rays 608 to visible light 610. An example scintillation screen 604 that may be used in a handheld X-ray scanner has a surface area of 4 inches by 6 inches. The size and material of the scintillation screen 604 at least partially determines the size, brightness, and/or resolution of the resulting digital images. The example scintillation screen is Gadox (Gadolinium oxysulphide) doped with Terbium, which emits a peak visible light at a wavelength of substantially 560 nm.

The example reflector 606 is a mirror that reflects visible light generated by the scintillation screen 604 to the digital imaging sensor 612 (e.g., via a lens 614). The example reflector 606 has the same surface area (e.g., 4 inches by 6 inches) as the scintillation screen 604, and is oriented at an angle 616 to direct the visible light 610 to the digital imaging sensor 612 and/or the lens 614. An example angle 616 is 30 degrees, which enables a 4 inch by 6 inch scintillation screen and a 4 inch by 6 inch reflector 606 to fit within a housing having a thickness 618 of less than 2.5 inches. In other examples, the angle 616 is an angle less than 45 degrees. Other sizes and/or geometries may be used for the scintillation screen 604 and/or the reflector 606. Additionally or alternatively, the digital X-ray detector 106 may include optics such as prisms to direct the visible light 610 to the digital imaging sensor 612.

The example digital imaging sensor 612 is a solid state sensor such as a CMOS camera. In the illustrated example using the scintillation screen 604 and the reflector 606, and a 6 mm lens 614, the digital imaging sensor 612 has a field of view of 143 degrees to capture light from substantially the entirety of the reflector 606.

The digital imaging sensor 612 is coupled to an imager bracket 620 via a mounting brackets 622. The detector housing 602 is also coupled to the imager bracket 620. The imager bracket 620 couples both the detector housing 602 and the digital imaging sensor 612 to the frame 102 of the handheld X-ray imaging system 100.

The mounting brackets 622 includes slots 624 to which an imaging bracket 626 is adjustably coupled. The digital imaging sensor 612 is attached to the imaging bracket 626 (e.g., via a printed circuit board). The imaging bracket 626 may be adjusted and secured along the length of the slots 624 to adjust an angle of the digital imaging sensor 612 relative to the reflector 606. The field of view of the digital imaging sensor 612 is oriented substantially perpendicularly to the scintillation screen, within the angular limits permitted using the slots 624 and the imaging bracket 626.

The example imager bracket 620 may include a data connector 628 (FIG. 8) to enable sufficient data throughput from the digital imaging sensor 612 to a computing device or other image display and/or image storage devices. An example data connector 628 may be a USB 3.0 connector to connect a USB 3.0 bus between the digital imaging sensor 612 and the receiving device. The USB 3.0 bus provides sufficient bandwidth between the digital X-ray imaging system 200 and the receiving device for high-definition video or better resolution.

While an example implementation of the X-ray detector 106 is described above, other example implementations of the X-ray detector 106 include using a solid state image sensor, such as a CMOS panel or a CCD panel, coupled directly to a scintillator. The CMOS panel produces digital images based on visible light generated by the scintillator, and outputs the digital images to the computing device 304.

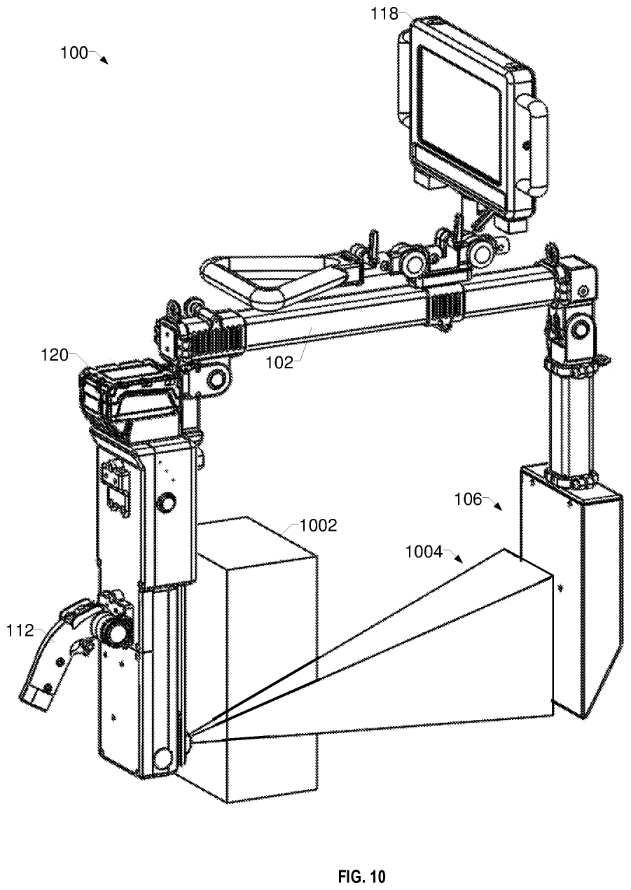

FIG. 10 is a side view of the handheld X-ray imaging system of FIG. 1, illustrating scanning of an object 1002 under test by directing X-rays 1004 from the X-ray tube 308 to the X-ray detector 106. As mentioned above, the collimator 310 reduces X-ray radiation that is not directed at the X-ray detector 106, so the concentration of the X-ray radiation 1004 that is not scattered by the object 1002 is incident on the X-ray detector 106.

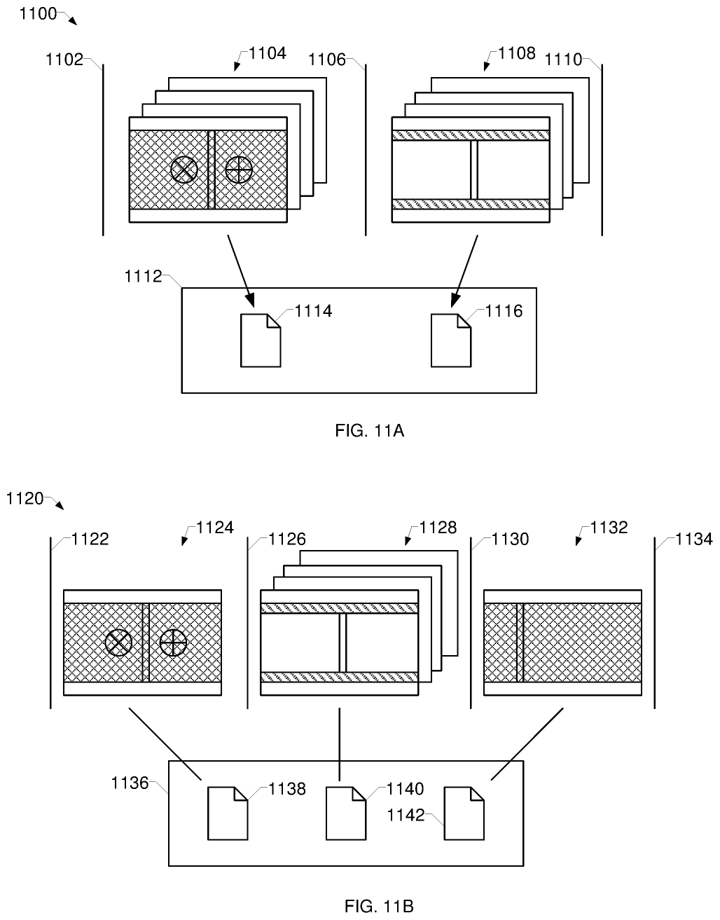

FIG. 11A illustrates example images and videos captured and stored, by the example handheld X-ray imaging system 100 of FIG. 1, as files in association with a recording session 1100. In the example of FIG. 11A, the recording session 1100 begins at time 1102 with a video 1104 containing a set of frames captured using the targeting camera(s) 236. The video 1104 is captured until a time 1106, at which time the operator actuates an input device to activate the X-ray output (e.g., the trigger 312). Following activation of the X-ray output at time 1106, a video 1108 containing a set of frames is captured using the X-ray detector 206. At time 1110, the recording session 1100 is ended.

The recording session 1100 is stored in a directory or folder file structure 1112, such as on the storage device(s) 244 of FIG. 2. Within the directory 1112, the video 1104 captured by the targeting camera(s) 236 is stored as a first file 1114 and the video 1108 captured by the X-ray detector 206 is stored as a second file 1116.

While the example recording session 1100 begins using the targeting camera(s) 236 and ends while using the X-ray detector 206, in other examples, recording sessions may begin using the X-ray detector 206 and end while using the targeting camera(s) 236.

FIG. 11B illustrates example images and videos captured and stored, by the example handheld X-ray imaging system 100 of FIG. 1, as files in association with another example recording session 1120. In the example of FIG. 11B, the recording session 1120 begins at a first time 1122 with a snapshot 1124 captured using the targeting camera(s) 236. In some examples, the computing device 208 may control the targeting camera(s) 236 to capture the snapshot 1124 and store the image automatically in response to starting the recording session 1120 while the X-ray output is active.

At time 1126, the operator actuates an input device to activate the X-ray output (e.g., the trigger 312). Following activation of the X-ray output at time 1126, a video 1128 containing a set of frames is captured using the X-ray detector 206. At time 1130, the X-ray output is deactivated (e.g., via the trigger 312), and the targeting camera(s) 236 are used to capture another image 1132. At time 1134, the recording session 1120 ends. In some examples, the computing device 208 may control the targeting camera(s) 236 to capture the snapshot 1132, store the snapshot 1132, and end the recording session 1120 automatically in response to deactivating the X-ray output during the recording session 1120.

The recording session 1100 is stored in a directory or folder file structure 1136, such as on the storage device(s) 244 of FIG. 2. Within the directory 1136, the snapshot 1124 captured by the targeting camera(s) 236 is stored as a first file 1138, the video 1128 captured by the X-ray detector 206 is stored as a second file 1140, and the snapshot 1132 captured by the targeting camera(s) 236 is stored as a third file 1142.

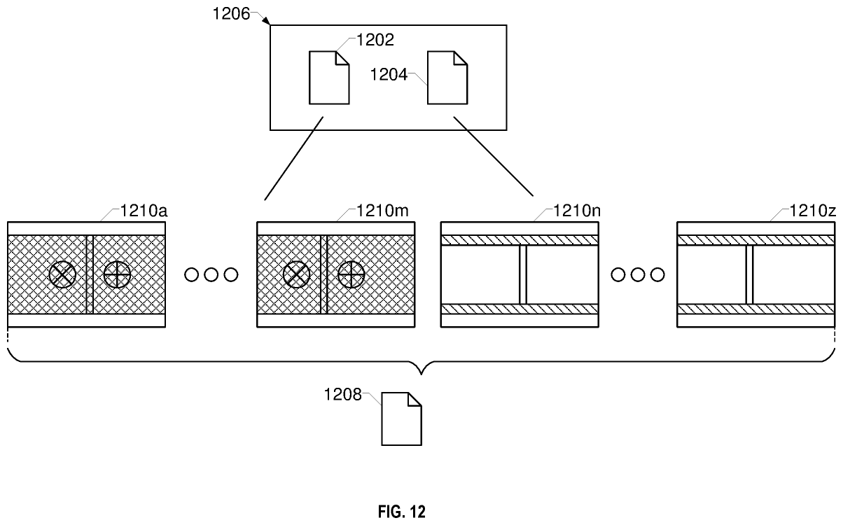

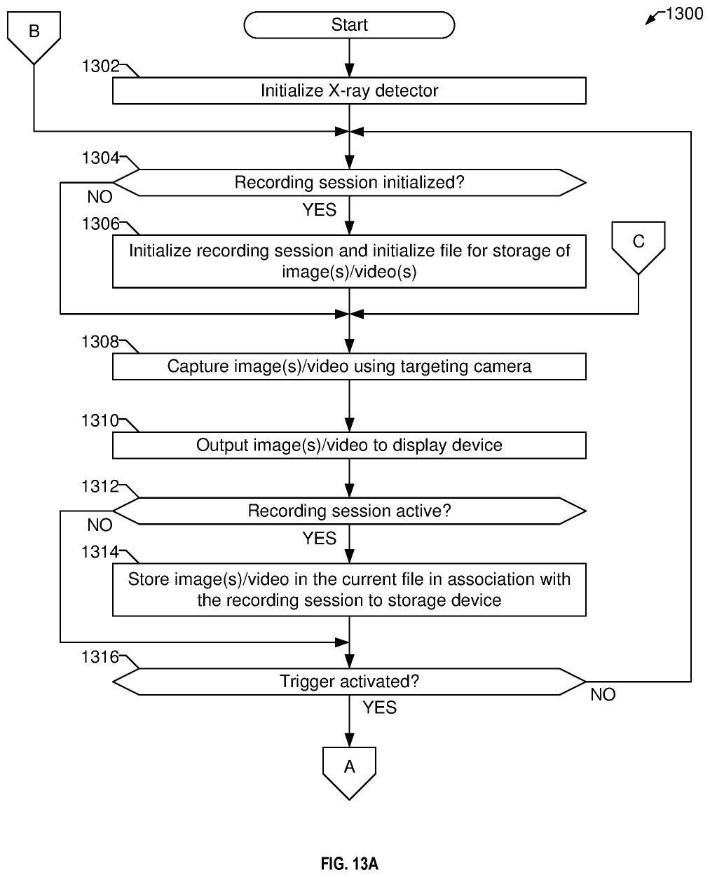

FIG. 12 illustrates an example of combining multiple files containing images and/or videos into a single, seamless video file captured by the example digital X-ray imaging system 200 of FIGS. 1 and 2. As illustrated in FIG. 12, multiple image and/or video files 1202, 1204 may be stored in a directory 1206 (e.g., in the storage device(s) 244, at the external computing device 238, etc.) that corresponds to a same recording session captured via the digital X-ray imaging system 200 using multiple imaging sensors (e.g., the targeting camera(s) 236 and the X-ray detector 206). The example external computing device 238 and/or the computing device 208 may combine the files 1202-1204 into a single (e.g., seamless) video file 1208 representative of the recording session.

To combine the files 1202-1204 into the single video file 1208, the example external computing device 238 splits up the video file 1202 into individual frames 1210a-1210m based on the number of frames in the video file 1202, and splits up the video file 1204 into individual frames 1210n-1210z based on the number of frames in the video file 1204. The external computing device 238 arranges the frames 1210a-1210z based on the timestamps associated with the video files 1202-1204 and/or associated with the frames 1210a-1210z to place the frames in sequential order. The external computing device 238 then combines the successive frames 1210a-1210z in sequence to create the seamless video and store the video in the single video file 1208.

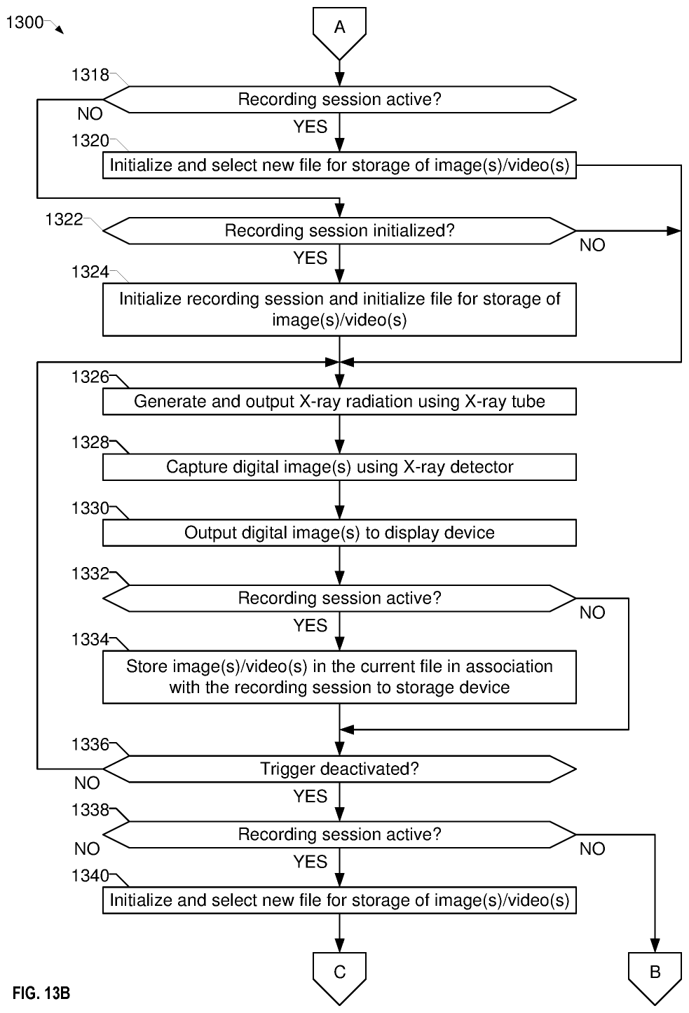

FIGS. 13A and 13B illustrate a flowchart representative of example machine readable instructions 1300 which may be executed by the example computing device of FIG. 2 to capture and store image(s) and/or video(s). The example machine readable instructions 1300 of FIGS. 13A-13B are described below with reference to the digital X-ray imaging system 200 of FIG. 2, but may be performed by the digital X-ray imaging system 100 of FIG. 1.

At block 1302, the example computing device 208 initializes the X-ray detector 206. For example, the computing device 208 may verify that the X-ray detector 206 is in communication with the computing device 208 and/or is configured to capture digital images of X-ray radiation.

At block 1304, the computing device 208 determines whether a recording session has been initialized. For example, the computing device 208 may detect one or more user inputs via the operator input device(s) 214, and/or identify an event or condition corresponding to automatically initializing a recording session. If a recording session has been initialized (block 1304), at block 1306 the computing device 208 initializes a recording session (e.g., the recording session 1100 or 1120 of FIGS. 11A and/or 11B), and initializes a file for storage of image(s) and/or video(s). For example, the computing device 208 may initialize a file for image(s) and video(s) to be stored as they are captured by the targeting camera(s) 236 and/or the X-ray detector 206.

After initializing the recording session (block 1306), or if a recording session is not initialized (block 1304), at block 1308 the computing device 208 captures image(s) and/or video using the targeting camera(s) 236. For example, the computing device 208 may receive a stream of one or more images captured by the targeting camera(s) 236. At block 1310, the computing device 208 outputs the image(s) and/or video to a display device, such as the display device(s) 212 and/or via the communications circuitry 240 to one or more external computing device(s) 238.

At block 1312, the computing device 208 determines whether a recording session is active. For example, a recording session may have been initialized at block 1304 and/or may already be active. If a recording session is active (block 1312), at block 1314 the computing device 208 stores the captured image(s) and/or video(s) in association with the recording session to the storage device(s) 244. In some examples, the computing device 208 may add auxiliary information such as location data and/or timestamps.

After storing the image(s)/video (block 1314), or if there is no active recording session (block 1312), at block 1316 the computing device 208 determines whether a trigger controlling the X-ray output is activated. For example, the computing device 208 may activate the X-ray tube 218 in response to activation of a trigger (e.g., a physical trigger, a button, a switch, etc.) by an operator. If the trigger is not activated (block 1316), control returns to block 1304 to determine whether a recording session is to be initialized.

Turning to FIG. 13B, when the trigger is activated (block 1316), the computing device 208 determines whether a recording session is already active. If a recording session is active (block 1318), at block 1320 the computing device 208 initializes and selects a new file for storage of image(s) and/or video from the X-ray detector 206. For example, the computing device 208 may stop recording to a file (e.g., the file 1114 of FIG. 11A) that was previously used for storing image(s) and/or video from the targeting camera(s) 236, and initializes a different file 1116 for storing image(s) and/or video from the X-ray detector 206.

Conversely, if a recording session is not active (block 1318), at block 1322 the computing device 208 determines whether a recording session has been initialized. Block 1322 may be similar or identical to block 1304 of FIG. 13A. If a recording session has been initialized (block 1322), at block 1324 the computing device 208 initializes a recording session (e.g., the recording session 1100 or 1120 of FIGS. 11A and/or 11B), and initializes a file for storage of image(s) and/or video(s). Block 1324 may be similar or identical to block 1306 of FIG. 13A.

After initializing a recording session (block 1324), initializing a file (block 1320), or if a recording session is neither active nor initialized (block 1318 and 1322), at block 1326 the X-ray tube 218 generates and outputs X-ray radiation. At block 1328, the X-ray detector 106 (e.g., via the scintillation screen 228, the reflector 230, and the digital imaging sensor 232, and/or via a solid state panel coupled to a scintillator) captures digital image(s) (e.g., digital still images and/or digital video). The X-ray detector 106 provides the captured digital image(s) to the computing device 208. At block 1330, the computing device 208 outputs the digital image(s) to the display device(s) 212 (e.g., via a wired and/or wireless connection).

At block 1332, the computing device 208 determines whether a recording session is active. If a recording session is active (block 1332), at block 1334 the computing device 208 stores the captured image(s) and/or video(s) in association with the recording session to the storage device(s) 244. In some examples, the computing device 208 may add auxiliary information such as location data and/or timestamps. Blocks 1332 and 1334 may be performed in a similar or identical manner as blocks 1312 and 1314.

After storing the image(s)/video (block 1334), or if there is no active recording session (block 1332), at block 1336 the computing device 208 determines whether the trigger has been deactivated. If the trigger is not deactivated (block 1336), control returns to block 1326 to continue capturing digital X-ray images.

When the trigger is deactivated (block 1336), at block 1338 the computing device 208 determines whether a recording session is already active. If a recording session is not active (block 1338), control returns to block 1304 of FIG. 13A. If a recording session is active (block 1338), at block 1340 the computing device 208 initializes and selects a new file for storage of image(s) and/or video from the targeting camera(s) 236. For example, the computing device 208 may stop recording to a file (e.g., the file 1116 of FIG. 11A) that was previously used for storing image(s) and/or video from the X-ray detector 206, and initializes a different file for storing image(s) and/or video from the targeting camera(s) 236. After initializing the new file (block 1340), control returns to block 1308 of FIG. 13A.

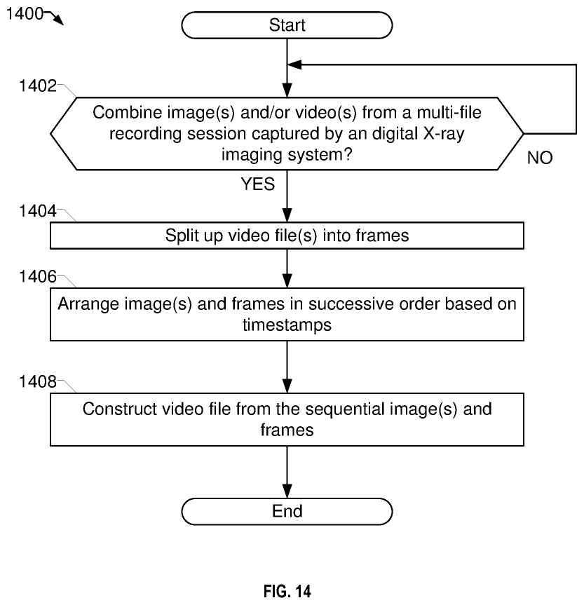

FIG. 14 is a flowchart representative of example machine readable instructions 1400 which may be executed by the example computing device 208 and/or the example external computing device 238 of FIG. 2 to combine multiple files (e.g., the files 1202-1204 of FIG. 12) into a single video file (e.g., the file 1208) representative of a recording session. The example instructions 1400 will be discussed below with reference to the example external computing device 238.

At block 1402, the external computing device 238 determines whether image(s) and/or video(s) from a multi-file recording session are to be combined. For example, the external computing device 238 combine files from a multi-file recording session as part of a synchronization process with the digital X-ray imaging system 200 and/or following transfer of the files to the external computing device 238. If no image(s) and/or video(s) from a multi-file recording session are to be combined (block 1402), control returns to block 1402 to await combination.

If image(s) and/or video(s) from a multi-file recording session are to be combined (block 1402), at block 1404 the external computing device 238 splits up video file(s) in a recording session into frames. For example, the external computing device 238 splits up the file 1202 into frames 1210a-1210m and splits up the file 1204 into frames 1210n-1210z.

At block 1406, the external computing device 238 arranges the image(s) and frames in successive order based on the timestamps. At block 1408, the external computing device 238 constructs the video file 1208 from the sequential image(s) and frames. The external computing device 238 stores the resulting video file 1208, and may delete the image(s) and/or video(s) used to generate the file 1208. The example instructions 1400 then end.

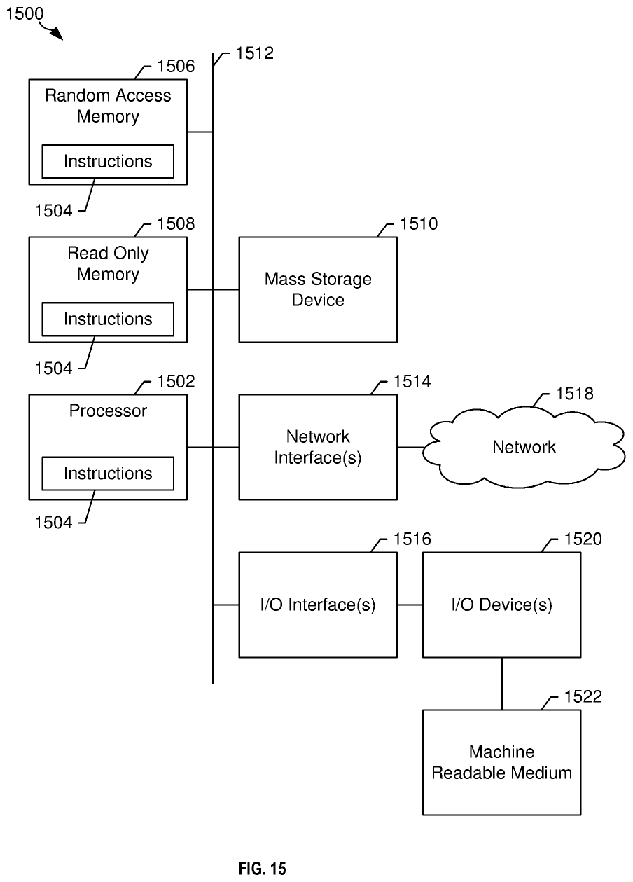

FIG. 15 is a block diagram of an example computing system 1500 that may be used to implement the computing device 208 and/or the external computing system(s) 238 of FIG. 2. The example computing system 1500 may be implemented using a personal computer, a server, a smartphone, a laptop computer, a workstation, a tablet computer, and/or any other type of computing device.

The example computing system 1500 of FIG. 15 includes a processor 1502. The example processor 1502 may be any general purpose central processing unit (CPU) from any manufacturer. In some other examples, the processor 1502 may include one or more specialized processing units, such as RISC processors with an ARM core, graphic processing units, digital signal processors, and/or system-on-chips (SoC). The processor 1502 executes machine readable instructions 1504 that may be stored locally at the processor (e.g., in an included cache or SoC), in a random access memory 1506 (or other volatile memory), in a read only memory 1508 (or other non-volatile memory such as FLASH memory), and/or in a mass storage device 1510. The example mass storage device 1510 may be a hard drive, a solid state storage drive, a hybrid drive, a RAID array, and/or any other mass data storage device.

A bus 1512 enables communications between the processor 1502, the RAM 1506, the ROM 1508, the mass storage device 1510, a network interface 1514, and/or an input/output interface 1516.

The example network interface 1514 (e.g., the communications circuitry 240) includes hardware, firmware, and/or software to connect the computing system 1500 to a communications network 1518 such as the Internet. For example, the network interface 1514 may include IEEE 1502. X-compliant wireless and/or wired communications hardware for transmitting and/or receiving communications.

The example I/O interface 1516 of FIG. 15 includes hardware, firmware, and/or software to connect one or more input/output devices 1520 to the processor 1502 for providing input to the processor 1502 and/or providing output from the processor 1502. For example, the I/O interface 1516 may include a graphics processing unit for interfacing with a display device, a universal serial bus port for interfacing with one or more USB-compliant devices, a FireWire, a field bus, and/or any other type of interface. Example I/O device(s) 1520 may include a keyboard, a keypad, a mouse, a trackball, a pointing device, a microphone, an audio speaker, an optical media drive, a multi-touch touch screen, a gesture recognition interface, a display device (e.g., the display device(s) 118, 212) a magnetic media drive, and/or any other type of input and/or output device.

The example computing system 1500 may access a non-transitory machine readable medium 1522 via the I/O interface 1516 and/or the I/O device(s) 1520. Examples of the machine readable medium 1522 of FIG. 15 include optical discs (e.g., compact discs (CDs), digital versatile/video discs (DVDs), Blu-ray discs, etc.), magnetic media (e.g., floppy disks), portable storage media (e.g., portable flash drives, secure digital (SD) cards, etc.), and/or any other type of removable and/or installed machine readable media.

Example wireless interfaces, protocols, and/or standards that may be supported and/or used by the network interface(s) 1514 and/or the I/O interface(s) 1516, such as to communicate with the display device(s) 212, include wireless personal area network (WPAN) protocols, such as Bluetooth (IEEE 802.15); near field communication (NFC) standards; wireless local area network (WLAN) protocols, such as WiFi (IEEE 802.11); cellular standards, such as 2G/2G+(e.g., GSM/GPRS/EDGE, and IS-95 or cdmaOne) and/or 2G/2G+(e.g., CDMA2000, UMTS, and HSPA); 4G standards, such as WiMAX (IEEE 802.16) and LTE; Ultra-Wideband (UWB); etc. Example wired interfaces, protocols, and/or standards that may be supported and/or used by the network interface(s) 1514 and/or the I/O interface(s) 1516, such as to communicate with the display device(s) 212, include comprise Ethernet (IEEE 802.3), Fiber Distributed Data Interface (FDDI), Integrated Services Digital Network (ISDN), cable television and/or internet (ATSC, DVB-C, DOCSIS), Universal Serial Bus (USB) based interfaces, etc.

The processor 1502, the network interface(s) 1514, and/or the I/O interface(s) 1516, and/or the display device 212, may perform signal processing operations such as, for example, filtering, amplification, analog-to-digital conversion and/or digital-to-analog conversion, up-conversion/down-conversion of baseband signals, encoding/decoding, encryption/decryption, modulation/demodulation, and/or any other appropriate signal processing.

The computing device 208 and/or the display device 212 may use one or more antennas for wireless communications and/or one or more wired port(s) for wired communications. The antenna(s) may be any type of antenna (e.g., directional antennas, omnidirectional antennas, multi-input multi-output (MIMO) antennas, etc.) suited for the frequencies, power levels, diversity, and/or other parameters required for the wireless interfaces and/or protocols used to communicate. The port(s) may include any type of connectors suited for the communications over wired interfaces/protocols supported by the computing device 208 and/or the display device 212. For example, the port(s) may include an Ethernet over twisted pair port, a USB port, an HDMI port, a passive optical network (PON) port, and/or any other suitable port for interfacing with a wired or optical cable.

The present methods and systems may be realized in hardware, software, and/or a combination of hardware and software. The present methods and/or systems may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may include a general-purpose computing system with a program or other code that, when being loaded and executed, controls the computing system such that it carries out the methods described herein. Another typical implementation may comprise an application specific integrated circuit or chip. Some implementations may comprise a non-transitory machine-readable (e.g., computer readable) medium (e.g., FLASH drive, optical disk, magnetic storage disk, or the like) having stored thereon one or more lines of code executable by a machine, thereby causing the machine to perform processes as described herein. As used herein, the term “non-transitory machine-readable medium” is defined to include all types of machine readable storage media and to exclude propagating signals.

As utilized herein the terms “circuits” and “circuitry” refer to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled or not enabled (e.g., by a user-configurable setting, factory trim, etc.).

While the present method and/or system has been described with reference to certain implementations, it will be understood by those skilled in the art that various changes may be made and equivalents may be substituted without departing from the scope of the present method and/or system. For example, block and/or components of disclosed examples may be combined, divided, re-arranged, and/or otherwise modified. In addition, many modifications may be made to adapt a particular situation or material to the teachings of the present disclosure without departing from its scope. Therefore, the present method and/or system are not limited to the particular implementations disclosed. Instead, the present method and/or system will include all implementations falling within the scope of the appended claims, both literally and under the doctrine of equivalents.

1. A method to generate a video from multiple cameras including a digital X-ray imaging device, the method comprising:
initiating a recording session on an X-ray digital imaging device;
capturing one or more images using a first one of a plurality of imaging sensors of the X-ray digital imaging device;
capturing one or more videos using a second one of the plurality of imaging sensors;
storing, on a machine readable storage device, two or more files corresponding to the one or more images and the one or more videos captured using the first and second ones of the plurality of imaging sensors; and
combining the two or more files into a single video file representative of the recording session.

2. The method as defined in claim 1, further comprising associating the two or more files with the same recording session, wherein the combining of the two or more files is based on the two or more files being associated with the same recording session.

3. The method as defined in claim 1, further comprising transmitting the two or more video files from the X-ray digital imaging device to an external computing device, wherein the external computing device automatically combines the two or more video files into the single video file.

4. The method as defined in claim 1, wherein capturing a first one of the one or more images using the first one of the plurality of imaging sensors is in response to initiating the recording session, and capturing a first one of the one or more videos using the second one of the plurality of imaging sensors is in response to actuating or deactuating an input device that controls the second one of the plurality of imaging sensors.

5. The method as defined in claim 1, wherein the plurality of imaging sensors comprises at least one of a visible light camera an infrared camera, or a thermal imaging camera, and a digital X-ray imaging sensor.

6. The method as defined in claim 5, wherein the at least one of the visible light camera or the infrared camera has a first field of view for the capturing of the one or more images using the first one of a plurality of imaging sensors, and the first field of view overlaps with a exposure field of an X-ray source that directs X-rays toward the digital X-ray imaging sensor for the capturing of the one or more videos using the second one of the plurality of imaging sensors.

7. The method as defined in claim 1, wherein the storing of the two or more files comprises storing timestamps associated with the two or more files, and the combining the two or more files into the single video file is based on the timestamps.

8. The method as defined in claim 1, wherein the storing the two or more files comprises:
storing a first video file or an image file captured via a targeting camera and in response to the initiation of the recording session, wherein the targeting camera is the first one of the plurality of imaging sensors; and
storing a second video file captured via a X-ray digital imaging sensor during the recording session and in response to receiving an input initiating output of X-rays, wherein the X-ray digital imaging sensor is the second one of the plurality of imaging sensors.

9. The method as defined in claim 1, wherein the storing the two or more files comprises:
storing a first video file captured via a X-ray digital imaging sensor during the recording session and in response to the initiation of the recording session, wherein the X-ray digital imaging sensor is the first one of the plurality of imaging sensors; and
storing a second video file or an image file captured via a targeting camera and in response to receiving an input ceasing output of X-rays, wherein the targeting camera is the second one of the plurality of imaging sensors.

10. The method as defined in claim 1, wherein the storing the two or more files comprises:
storing a first video file or first image file captured via a targeting camera and in response to the initiation of the recording session, wherein the targeting camera is the first one of the plurality of imaging sensors;
storing a second video file captured via a X-ray digital imaging sensor during the recording session and in response to receiving a first input initiating output of X-rays, wherein the X-ray digital imaging sensor is the second one of the plurality of imaging sensors; and
storing a third video file or second image file captured via the targeting camera during the recording session and in response to receiving a second input ceasing output of X-rays.

11. The method as defined in claim 1, wherein the one or more images comprise at least one of an image or a video.

12. An X-ray scanning system, comprising:
a portable X-ray scanner, comprising:
a digital X-ray detector configured to generate digital video based on incident X-ray radiation;
an X-ray tube configured to output X-ray radiation;
a targeting camera configured to generate digital video of at least a portion of an exposure field of the X-ray tube; and
a machine readable storage device; and
one or more computing devices configured to:
control the X-ray tube;
initiate a recording session on the portable X-ray scanner;
capture one or more videos using a first one of a plurality of imaging sensors of the targeting camera;
capture one or more videos using the digital X-ray detector;
store, on the machine readable storage device, two or more files corresponding to the videos captured using the first and second ones of the plurality of imaging sensors; and
combine the two or more files into a single video file representative of the recording session.

13. The X-ray scanning system as defined in claim 12, wherein the one or more computing devices comprise an external computing device separate from the portable X-ray scanner configured to combine the two or more files into the single video file representative of the recording session.

14. The X-ray scanning system as defined in claim 13, wherein the portable X-ray scanner comprises a second one of the one or more computing devices, configured to:
control the X-ray tube;
initiate the recording session on the portable X-ray scanner;
capture the one or more images using the first one of a plurality of imaging sensors of the portable X-ray scanner;
capture the one or more videos using the second one of the plurality of imaging sensors;
store the two or more files corresponding to the one or more images and one or more videos captured using the first and second ones of the plurality of imaging sensors; and
transmit the two or more files to the external computing device.

15. The X-ray scanning system as defined in claim 12, further comprising one or more input devices configured to change between the first one of the plurality of imaging sensors and the second one of the plurality of imaging sensors for capturing video.

16. The X-ray scanning system as defined in claim 15, wherein the one or more computing devices are configured to:
store a first video file or a first image file captured via the targeting camera and in response to the initiation of the recording session; and
store a second video file captured via the digital X-ray detector during the recording session and in response to the one or more input devices.

17. The X-ray scanning system as defined in claim 15, wherein the one or more computing devices are configured to:
store a first video file captured via the digital X-ray detector during the recording session and in response to the initiation of the recording session; and
store a second video file or image file captured via the targeting camera and in response to the one or more input devices.

18. The X-ray scanning system as defined in claim 14, wherein the one or more computing devices are configured to:
store a first video file or a first image file captured via the targeting camera and in response to the initiation of the recording session;
store a second video file captured via the digital X-ray detector during the recording session and in response to the one or more input devices; and
storing a third video file or a second image file captured via the targeting camera during the recording session and in response to the one or more input devices.

19. The X-ray scanning system as defined in claim 11, wherein the one or more computing devices are configured to store timestamps associated with the two or more files, and combine the two or more files into the single video file based on the timestamps.

20. The X-ray scanning system as defined in claim 11, wherein the one or more images comprise at least one of an image or a video.
